(19) United States
(12) Patent Application Publication
(10) Pub. No.: US 2003/0215413 A1
(43) Pub. Date: Nov. 20, 2003

(54) Compositions containing stabilized retinoid

(75) Inventors: Hani Fares, Somerset, NJ (US); Sidney P. Foltis, Nutley, NJ (US); Thomas Re, Clark, NJ (US); Alan Meyers, Clark, NJ (US); Mark Cornell, Clark, NJ (US); Isabelle Hansenne, Clark, NJ (US)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/393,923

(22) Filed: Mar. 24, 2003

Related U.S. Application Data
(60) Provisional application No. 60/366,235, filed on Mar. 22, 2002.

(51) Int. Cl. A61K 7/06; A61K 7/11; A61K 31/11; A61K 31/07; A61K 31/203

(52) U.S. Cl. 424/70.16; 514/559; 514/725; 514/703

(57) ABSTRACT

The present invention provides compositions useful for skin and/or hair care that contain a retinoid in which the retinoid remains stable over a sustained period of time.

### CROSS-REFERENCE TO A RELATED APPLICATIONS

This application claims the benefit of U.S. application Ser. No. 60/366,235 filed on Mar. 22, 2002.

### FIELD OF THE INVENTION

The present invention provides compositions useful for skin and/or hair care that contain a retinoid in which the retinoid remains stable over a sustained period of time.

### DISCUSSION OF THE BACKGROUND

Cosmetic and/or dermatological compositions containing retinoids have undergone considerable development in recent years. Compositions containing retinoids can be used for numerous cosmetic and/or dermatological purposes such as, for example, treating acne, rosacea, or hyperpigmentation, and reducing the effects associated with aging such as wrinkles, a weather-beaten appearance, yellowing, the loss of elasticity, redness, dryness, and age spots. Such manifestations associated with aging are proportionately more pronounced when the skin has been frequently exposed to the sun or is particularly sensitive to exposure to UV radiation.

Thus, the effects of intrinsic ageing of the skin (associated with age) and of light-induced ageing (due to exposure to the sun), chrono-ageing, and photo-ageing can be cumulative. The manifestations of ageing usually appear at an advanced age; however, their prevention should be undertaken from the start of adult life or earlier, by means of appropriate care measures.

Treatment of the skin with derivatives of the retinoid family forms a part of these preventive or curative measures for caring for the manifestations of ageing, namely: wrinkles, weather-beaten skin, yellowing, loss of elasticity, redness, dryness, and marks.

Among the retinoids, retinol, also known as vitamin A, and esterified derivatives of retinol are most particularly advantageous for treating and/or reducing skin conditions. In point of fact, retinol is a natural endogenous constituent of the human body. It is well tolerated when applied to the skin, up to levels much higher than those for retinoic acid. Retinol esters are converted into retinol by the human body.

However, when they are introduced into a cosmetic or dermatological composition intended for topical application, they rapidly degrade, under the effect of light, oxygen, metal ions, oxidizing agents, water or, in particular, under the effect of an increase in temperature. The thermal degradation of retinol was the subject of a study published in J. Soc. Cosm. Chem. 46, 191-198 (July-August 1995).

However, there remains a need in the art for stable retinoid compositions.

### SUMMARY OF THE INVENTION

Therefore, an object of the present invention is to provide compositions containing a retinoid in which the retinoid remains stable over a sustained period of time. It is also an object of the present invention to provide methods for ameliorating or reducing the signs of aging comprising applying a composition containing a retinoid in which the retinoid remains stable over a sustained period of time to the skin in an ameliorating- or reducing-effective amount.

It is a further object of the present invention to provide methods for reducing or ameliorating the irritative effects associated with application of high levels of retinoid to the skin.

### DETAILED DESCRIPTION OF THE INVENTION

Retinoids useful in the compositions of the present invention include retinol (vitamin A), a biological precursor of retinol, a chemical derivative of retinol, and a metabolic derivative of retinol. The retinoid can be water-soluble or oil-soluble. Specifically included within this definition are, for example, retinoic acid, retinal (retinol aldehyde), retinol esters, in particular, C₁-C₁₂ esters such as retinyl acetate, retinyl propionate, retinyl butylate, retinyl hexanoate, retinyl heptanoate, retinyl caprylate, and retinyl palmitate. Preferred retinoids include retinol, retinoic acid, retinal, retinyl acetate, retinyl caprylate and retinyl palmitate.

In the compositions according to the present invention, the retinoid is preferably present in an amount ranging from 0.001 to 10% by weight relative to the total weight of the composition, advantageously from 0.01 to 3% and even more preferably from 0.1 to 1%, with 0.15 to 0.75 being most preferred. These weight % ranges include all specific values and subranges there between, such as 0.1, 0.15, 0.2, 0.25, 0.3 0.5, 0.6, 2, 3, 5 and 8% by weight.

The compositions of the present invention enable the formulation of relatively high concentrations of retinoids without subjecting the user of the composition to the irritative effects normally associated with application of high levels of retinoid to skin. In other words, the compositions of the present invention avoid, reduce, and/or ameliorate the irritating effects normally associated with the application of retinoids to skin. Due to the reduced irritation, greater amounts of retinoid can be provided in the compositions which, in turn, allow greater delivery of retinoid to the user's skin and/or hair as described herein. “High concentrations of retinoids” means 0.1% by weight relative to the total weight of the composition or greater. Stabilized retinoids as used herein means that the retinoid is subject to little or no degradation upon storage for extended periods of time. For example, the amount of retinoid remaining in the composition after storage for 4, 6, 8, 10, 12, 16, 20, 24 or more weeks, will be at least 85% of the original amount, preferably 90% of the original amount, more preferably 95%, inclusive of 86, 87, 88, 89, 91, 92, 93, 94, 96, 97, 98, 99, and 100% of the retinoid remaining after the period of time. In one embodiment, the retinoid is stable at various temperatures, including 4, 15, 20, 25, 35, 40, and 45° C.

According to one embodiment of the present invention, a composition comprising a retinoid and a polymer is provided, wherein the retinoid remains stable in the composition for a sustained period of time.

One skilled in the art will be able, by means of simple tests, to adapt the relative proportion of retinoid and polymer. The optimum proportions of the various constituents can vary, for example, as a function of the molecular weight of the polymer, and/or other physical properties of the polymer.

Exemplary crosslinked polymers of at least one ethylenically unsaturated monomer according to the invention include, in particular:

(i) the crosslinked homopolymers or copolymers of at least one ethylenically unsaturated monomer having a sulfonic functional group, in free or partially or completely salified form;

(ii) the crosslinked copolymers of at least one ethylenically unsaturated monomer having a carboxylic acid functional group, in free or partially or completely salified form, or of an ester or amide derivative thereof;

(iii) the crosslinked homopolymers or copolymers of at least one cationic ethylenically unsaturated monomer of the ester or amide type.

The monomers having a sulfonic acid function are selected, in particular, from among 2-acrylamido-2-methylpropanesulfonic acid as well as the salts thereof.

The monomers having a carboxylic acid function are selected, in particular, from among acrylic acid and methacrylic acid as well as the salts thereof. Their esters are advantageously selected from among those of (meth)acrylic acid and C₁-C₃₀ alcohols. Their amides are advantageously selected from among those of (meth)acrylic acid and C₁-C₃₀ amines and, more particularly, methacrylamide and/or acrylamide.

The cationic monomers of the ester or amide type are preferably selected from among ammonium (meth)acrylate, dialkylaminoalkyl (meth)acrylates, in particular dimethylaminoethyl methacrylate; dialkylaminoalkyl (meth)acrylamides as well as their quaternary salts or acids; the alkyl radicals preferably being C₁-C₄.

The crosslinked polymers of the invention comprise the polymerizates of at least one ethylenically unsaturated crosslinking agent which is preferably selected from among divinylbenzene; tetraallyloxyethane; diallyl ether; polyallyl polyglyceryl ethers; allyl ethers of alcohols of the sugar series such as erythritol, pentaerythritol, arabitol, sorbitol or glucose; methylenebisacrylamide, ethylene glycol di(methyl)acrylate, di(meth)acrylamide, cyanomethyl acrylate, vinyloxethyl (meth)acrylate and their metal salts.

Exemplary crosslinked homopolymers or copolymers of at least one ethylenically unsaturated monomer having a sulfonic function, which are at least 90% neutralized, more particularly include the crosslinked homopolymers of 2-acrylamido-2-methylpropanesulfonic acid, which are at least 90% neutralized, such as those described in EP-A-0,815,828 (hereby expressly incorporated by reference), the crosslinked copolymers of 2-acrylamido-2-methylpropanesulfonic acid and of acrylamide which are partially or completely neutralized (with a base such as sodium hydroxide, potassium hydroxide or an amine) as described in EP-A-0,503,853 (also expressly incorporated by reference) and more particularly in Example 1 thereof.

Exemplary crosslinked copolymers of at least one ethylenically unsaturated monomer having a carboxylic acid function or of an ester or amide derivative thereof, more particularly include the non-ionic or anionic amphiphilic polymers comprising at least one fatty chain or substituent and at least one hydrophilic structural unit.

The non-ionic amphiphilic polymers are advantageously selected from among:

(i) the copolymers of C₁-C₆ alkyl methacrylates or acrylates and of amphiphilic monomers comprising at least one fatty chain such as, for example, the oxyethylenated stearyl acrylate/methyl acrylate copolymer marketed by Goldschmidt under the trademark ANTIL 208;

(ii) the copolymers of hydrophilic methacrylates or acrylates and of hydrophobic monomers comprising at least one fatty chain such as, for example, the polyethylene glycol methacrylate/lauryl methacrylate copolymer.

The anionic amphiphilic polymers are advantageously selected from among those in which the hydrophilic structural unit is an ethylenically unsaturated anionic monomer, more particularly of a vinylcarboxylic acid and most particularly of an acrylic acid, a methacrylic acid or mixture thereof, and also comprising an allyl ether structural unit containing a fatty chain which corresponds to a monomer having the following formula (I):

CH₂═C(R′)CH₂OBₙR (I)

in which R′ is H or CH₃, B represents the ethyleneoxy radical, n is zero or an integer ranging from 1 to 100, R is a hydrocarbon radical selected from among alkyl, arylalkyl, aryl, alkylaryl or cycloalkyl radicals, having from 8 to 30 carbon atoms, preferably 10 to 24, and more preferably from 12 to 18 carbon atoms. A more particularly preferred structural unit of formula (I) is a structural unit in which R′ is H, n is equal to 10, and R is a stearyl (C₁₈) radical. Anionic amphiphilic polymers of this category are described and prepared, via technique for polymerization in emulsion, in EP-0,216,479 B2.

Exemplary of these associative anionic polymers are those polymers prepared from 20% to 60% by weight of acrylic acid and/or of methacrylic acid, from 5% to 60% by weight of lower alkyl(meth)acrylates, from 2% to 50% by weight of allyl ether bearing a fatty chain substituent of formula (I), and from 0% to 1% by weight of a crosslinking agent which is a well known copolymerizable polyethylenic unsaturated monomer such as diallyl phthalate, allyl (meth)acrylate, divinylbenzene, (poly)ethylene glycol dimethacrylate and methylenebisacrylamide. There are most particularly preferred according to the invention.

Among the latter, the crosslinked terpolymers of methacrylic acid, ethyl acrylate, polyethylene glycol (10 EO) stearyl alcohol ether (Steareth 10), in particular those marketed by Allied Colloids, under the trademarks SALCARE SC 80 and SALCARE SC 90 which are aqueous emulsions containing 30% of a crosslinked terpolymer of methacrylic acid, of ethyl acrylate and of steareth-10-allyl ether (40/50/10) are most particularly preferred.

Exemplary associative anionic polymers include those anionic polymers comprising at least one hydrophilic structural unit of the olefinic unsaturated carboxylic acid type, and at least one hydrophobic structural unit exclusively of the (C₁₀-C₃₀)alkyl ester of unsaturated carboxylic acid type.

Preferably, these polymers are selected from among those whose hydrophilic structural unit of the olefinic unsaturated carboxylic acid type corresponds to the monomer having the following formula (II):

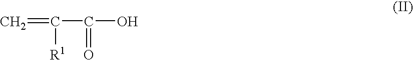

in which R₁ is H or CH₃ or C₂H₅, namely, acrylic acid, methacrylic acid or ethacrylic acid structural units, and whose hydrophobic structural unit of the (C₁₀-C₃₀)alkyl ester of unsaturated carboxylic acid type corresponds to the monomer having the following formula (III):

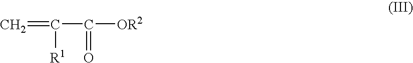

in which R₂ is H or CH₃ or C₂H₅ (namely, acrylate, methacrylate or ethacrylate structural units) and preferably H (acrylate units) or CH₃ (methacrylate units), and R₃ is a C₁₀-C₃₀, and preferably C₁₂-C₂₂, alkyl radical.

(C₁₀-C₃₀)alkyl esters of unsaturated carboxylic acids in accordance with the invention include, for example, lauryl acrylate, stearyl acrylate, decyl acrylate, isodecyl acrylate and the corresponding methacrylates, lauryl methacrylate, stearyl methacrylate, decyl methacrylate and isodecyl methacrylate.

Anionic polymers of this type are for example described and prepared according to the methodology set forth in U.S. Pat. Nos. 3,915,921 and 4,509,949.

Exemplary of the above polymers are the products marketed by Noveon, Inc. under the trademark PEMULEN TR1, PEMULEN TR2, which are acrylates/C₁₀-C₃₀ alkyl acrylate copolymers, CARBOPOL 1382, which is a carboxyvinyl polymer (INCI name: acrylates/C₁₀-C₃₀ alkyl acrylate copolymers), CARBOPOL 980, which is a homopolymer of acrylic acid, cross-linked with an allyl ether of an allyl ether of pentaerythritol, an allyl ether of sucrose, or an allyl ether of propylene (also known as Carbomer according to CFTA designation) and more preferably PEMULEN TR1, and the product marketed by S.E.P.P.I.C. under the trademark COATEX SX. The copolymers sold under the name Pemulen by Noveon, Inc. and in particular the, such as the product Pemulen TR1 or Pemulen TR2 are preferably used.

Other polymers, which can be used include polyacrylodimethyl taurate, Simulgel 600™ (copolymer of acrylamide/acryloyldimethyltaurate copolymer (and) isohexadecane (and) polysorbate 80); and Sepigel 305 (sold by Seppic), which is an inverse emulsion of crosslinked copolymer obtained from acrylamide and from the sodium salt of 2-acrylamido-2-propanesulphonic acid (see also EP 0503853, which is incorporated by reference herein). These are most particularly preferred according to the present invention.

Exemplary crosslinked homopolymers or copolymers of at least one cationic ethylenically unsaturated monomer of the ester or amide type, include, in particular:

(i) the homopolymers of ammonium acrylate or the copolymers of ammonium acrylate and of acrylamide, such as the product marketed under the trademark BOZEPOL C NOUVEAU or the product PAS 5193 marketed by Hoechst (described and prepared as set forth in FR-2,416,723, and U.S. Pat. Nos. 2,798,053 and 2,923,692);

(ii) the homopolymers of dimethylaminoethyl methacrylate quaternized with methyl chloride, such as the products marketed under the trademarks SALCARE 95 and SALCARE 96 by Allied Colloids, or the copolymers of dimethylaminoethyl methacrylate quaternized with methyl chloride and of acrylamide, such as the product SALCARE SC92 marketed by Allied Colloids or the product PAS 5194 marketed by Hoechst (described and prepared as set forth in EP-A-0,395,282).

The above polymers according to the invention are generally formulated into the subject compositions in a concentration (expressed as active substance AS) ranging from 0.01% to 10% by weight, and preferably from 0.1% to 5% by weight, relative to the total weight of the composition.

Mixtures of several copolymers, as defined above, i.e., more than one, may be used.

The compositions of the present invention contain in addition to or instead of the polymers discussed above, one or more thickening and gelling agents, which include materials which are primarily derived from natural sources. Nonlimiting examples of these gelling agent gums include materials selected from the group consisting of acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, anatto gum, potassium alginate, potassium carrageenan, propylene glycol alginate, sclerotium gum, sodium carboxymethyl dextran, sodium carboxylmethyl cellulose, sodium carrageenan, tragacanth gum, xanthan gum, and mixtures thereof. Preferred are xanthan and gellan gum.

Other additional thickening and gelling agents useful herein include crosslinked copolymers of alkyl vinyl ethers and maleic anhydride. In these copolymers the vinyl ethers are represented by the formula R—O—CH═CH₂ wherein R is a C₁-C₆ alkyl group, preferably R is methyl. Preferred crosslinking agents are C₄-C₂₀ dienes, preferably C₆ to C₆ dienes, and most preferably C₈ to C₁₂ dienes. A particularly preferred copolymer is one formed from methyl vinyl ether and maleic anhydride wherein the copolymer has been crosslinked with decadiene, and wherein the polymer when diluted as a 0.5% aqueous solution at pH 7 at 25° C. has a viscosity of 50,000-70,000 cps when measured using a Brookfield RTV viscometer, spindle #7 at 10 rpm. This copolymer has the INCI designation PVM/MA decadiene crosspolymer and is commercially available as Stabileze.TM. 06 from International Specialty Products (Wayne N.J.).

Crosslinked polyvinyl(N-pyrrolidones) useful herein as additional thickening and gelling agents and include those described in U.S. Pat. No. 5,139,770 and U.S. Pat. No. 5,073,614, which are incorporated by reference herein. These gelling agents typically contain from about 0.25% to about 1% by weight of a crosslinking agent selected from the group consisting of divinyl ethers and diallyl ethers of terminal diols containing from about 2 to about 12 carbon atoms, divinyl ethers and diallyl ethers of polyethylene glycols containing from about 2 to about 600 units, dienes having from about 6 to about 20 carbon atoms, divinyl benzene, vinyl and allyl ethers of pentaerythritol, and the like. Typically, these gelling agents have a viscosity from about 25,000 cps to about 40,000 cps when measured as a 5% aqueous solution at 25° C. using a Brookfield RVT viscometer with Spindle #6 at 10 rpm. Commercially available examples of these polymers include ACP-1120, ACP-1179, and ACP-1180, available from International Specialty Products (Wayne, N.J.).

Thickening agents which are suitable for use herein also include those disclosed in U.S. Pat. No., 4,387,107, and “Encyclopedia of Polymers and Thickeners for Cosmetics,” R. Y. Lochhead and W. R. Fron, eds., Cosmetics & Toiletries, vol. 108, pp. 95-135 (May 1993), which are incorporated herein by reference.

These thickeners can be present in the compositions according to the invention in an amount ranging from 0.01 to 3% by weight with respect to the total weight of the composition, preferably 0.02 to 0.6% by weight and more preferably from 0.05 to 0.2% by weight. These ranges for amount of thickeners in the composition include all specific values and subranges there between, such as 0.1, 0.5, 1, 1.5, 2 and 2.5% by weight with respect to the total weight of the composition.

Oils which can be used in the present invention include, but are not limited to: oils of vegetable origin, such as liquid triglycerides, for example sunflower, maize, soybean, jojoba, cucumber, grape seed, sesame, hazelnut, apricot, macadamia or castor oil; or triglycerides of caprylic/capric acids, such as those sold by the company Stéarineries Dubois or those sold under the names Miglyol 810, 812 and 818 by the company Dynamit Nobel, oils of animal origin, such as lanolin, oils of mineral origin, synthetic oils, such as fatty alcohols, for example 2-octyldodecanol; esters and in particular fatty acid esters and especially esters having a total number of carbon atoms chosen between 12 and 80, preferably between 16 and 50; or phenylated silicones and in particular phenyl trimethicones, diphenyl dimethicones or polymethylphenylsiloxanes.

When the composition is an emulsion, the composition can comprise 1 to 50% by weight of fatty phase, preferably 5 to 30% by weight and more preferably 10 to 20% by weight of fatty phase.

As known in the art, the emulsion according to the invention can optionally comprise a small amount of a surfactant, in particular an O/W surfactant, although this is not necessary to obtain a stable emulsion. The amount of surfactant can represent from 0.1 to 3% and preferably from 0.1 to 2% of the total weight of the emulsion. Advantageously, the compositions of the present invention do not comprise surfactants.

The compositions according to the invention can be used in particular in the cosmetics and/or dermatological fields. It can be used as is and thus itself constitute a cosmetic or dermatological composition; it can also be incorporated in a more sophisticated cosmetic or dermatological composition.

The compositions according to the invention can be formulated into a cosmetically or dermatologically acceptable medium, i.e., a medium compatible with all keratinous substances, such as the skin, nails, mucous membranes and hair or any other cutaneous region of the body. This medium can comprise, as known in the art, constituents conventionally employed in the cosmetic and/or dermatologic fields.

The compositions of the present invention can be used in a great number of cosmetic or dermatological treatments of the skin, hair, eyelashes, eyebrows, nails, mucous membranes or scalp; for example face and/or body care (protection, treatment or care creams for the face, for the hands or for the body; protection or care body milks; or lotions, gels or foams for caring for the skin and mucous membranes or for cleansing the skin); making up the face and/or body (lipstick, eyeliner, foundation, mascara, concealer, eye shadow or face powder); removing make-up; antisun protection; or the dermatological treatment of diseases of the skin, hair, eyelashes, eyebrows, nails, scalp and/or mucous membranes.

The compositions of the present invention should be typically formulated in a pH of from about 4 to about 10 to prevent any irritation or discomfort to the user when the pH is too acidic or basic. This pH range includes, 4.5, 5.0, 5.5, 6.0, 6.5, 7.0, 7.5, 8.0, 8.5, 9.0 and 9.5.

Additional optional components include, but are not limited to, waxes chosen from animal, fossil, vegetable, mineral or synthetic waxes known per se, such as paraffin waxes, polyethylene waxes, carnauba or candelilla waxes, beeswaxes, microcrystalline wax or silicone waxes; pulverulent materials, such as fillers, pigments and/or pearlescent agents, ultraviolet light filters, dyes, organic solvents, organic diluents, chelating agents, pH adjusting agents, conditioning agents, humectants, lipids, fragrances, preservatives, proteins, protein derivatives, amino acids, amino acid derivatives, other skin active agents, suspending agents, sunscreens, thickeners, vitamins, ceramide, uv absorbers (e.g., benzophenone), botanicals, anti-oxidants, and viscosity adjusting agents. These and other cosmetic additives commonly used in personal care formulations are described in, for example, International Cosmetic Ingredient Dictionary and Handbook (7^{th} Ed.) vol. 1-3 (1997), The Cosmetic, Toiletry and Fragrance Association, Washington, D.C.

These additional components, depending on their nature, can be introduced into the fatty phase or into the aqueous phase. Of course, a person skilled in the art will take care to choose this or these optional additional compounds and/or their amount so that the advantageous properties of the composition according to the invention are not, or not substantially, detrimentally affected by the envisaged addition.

These additional components can be present in the compositions in amount ranging from about 0.0001 to about 25% by weight, inclusive of all values and subranges there between.

The compositions can be made according to standard protocols known in the art for making emulsions, dispersions, and the like. In one embodiment a dispersion containing the retinoid is prepared under an inert gas, such as argon, away from fluorescent light and preferably under actinic lights. The process of producing such a dispersion can include the addition of the retinoid dissolved in oil, such as soybean oil, and a polymeric stabilizer.

In view of the above, one aspect of the present invention is a composition comprising a retinoid and a polymer or copolymer, wherein the composition retains at least about 90% of the retinoid after 12 weeks of storage at 45° C. In an alternate embodiment, the composition has at least 0.1% of retinoid and a polymer, wherein the composition retains at least about 90% of the retinoid after 12 weeks of storage at 45° C., and which has reduced irritation when applied to the skin.

The composition may be an emulsion or a dispersion. The composition may also be devoid of a surfactant and/or contain an antioxidant, one or more thickeners, one or more polymers such as carboxyvinyl polymer, crosslinked copolymer of acrylamide and the sodium salt of 2-acrylamido-2-propanesulphonic acid, a copolymer of monoolefinically unsaturated C₃-C₆ carboxylic acid, a copolymer of acid anhydride and an acrylic acid ester containing a fatty chain having from 10 to 30 carbon atoms, or mixtures thereof; a chelating agent.

In one embodiment, the composition comprises about 2.0% to about 7.0% Glycerin, including 2.5, 3.0, 3.5, 4.0, 4.5, 5.0, 5.5, 6.0, and 6.5%; about 0.5% to about 5.0%, Simulgel 600, including 1.0, 1.5, 2.0. 2.5, 3.0, 3.5, 4.0, and 4.5%; about 1.0% to about 7.0% Isostearyl neopentanoate, including 1.5, 2.0. 2.5, 3.0, 3.5, 4.0, 4.5, 5.0, 5.5, 6.0, and 6.5%; about 0.25% to about 2.0% Stearyl alcohol, including 0.5, 0.75, 1.0, 1.25, 1.5 and 1.75 %; about 0.5% to about 6.0% Retinol in soybean oil 10%; TEA as need to a pH of about 7; preservative; and water.

In another embodiment, the retinoid is added to a formulation of about 2.0% to about 7.0% Glycerin, including 2.5, 3.0, 3.5, 4.0, 4.5, 5.0, 5.5, 6.0, and 6.5%; about 0.05% to about 0.75% Pemulen TR-2, including about 0.1, 0.15, 0.2, 0.25, 0.3, 0.4, 0.5, 0.6 and 0.7%; about 0.05% to about 0.75% Carbopol 980, including 0.1, 0.15, 0.2, 0.25, 0.3, 0.4, 0.5, 0.6 and 0.7% ; about 1.0% to about 6.0% Capric/Caprylic Triglyceride, including 1.5, 2.0, 2.5, 3.0, 3.5, 4.0, 4.5, 5.0, 5.5, 6.0, and 6.5%; about 1.0% to about 8.0% Meadowfoam seed oil, including 1.5, 2.0, 2.5, 3.0, 3.5, 4.0, 4.5, 5.0, 5.5, 6.0, 6.5 and 7.0%; about 0.25% to about 2.0% Cetearyl alcohol, including 0.5, 0.75, 1.25, 1.5, and 1.75%; about 0.25% to about 1.0% Behehyl alcohol, including 0.5, 0.75, and 0.9%; about 0.25% to about 2.0% Triethanolamine, including 0.5, 0.75, 1.25, 1.5, and 1.75% and a preservative as desired.

Based on the Inventors' discovery that the retinoid compositions are stable and non-irritating, the present the invention provides methods of making the retinoid containing compositions, for example, by combining the ingredients under an inert gas, such as argon. Such a method provides a means to stabilize retinoids against thermal degradation, comprising combining a polymer or copolymer in an amount effective to stabilize the retinoid, wherein the composition retains at least about 90% of the retinoid after 12 weeks of storage at 45° C.

A subject of the invention is also a cosmetic treatment process which comprises reducing or ameliorating the effects associated with aging such as wrinkles, weather-beaten skin, yellowing, loss of elasticity, redness, dryness and marks by topical application to the skin and/or the scalp and/or the hair of a composition according to the invention. These effects also include photoperoxidation, in particular the photoperoxidation of squalene and/or collagen. The treatment protocol can ameliorate and/or reduce these signs of ageing. A subject of the invention is also the use of such a cosmetic composition for controlling and/or preventing the signs of light-induced ageing of the skin and/or the hair. A subject of the invention is also a dermatological composition comprising a combination according to the invention, which is intended for controlling the signs of ageing of the skin or the hair, particularly the signs of ageing induced by photoperoxidation, in particular the photoperoxidation of squalene and/or collagen. Therefore, the present invention provides methods of treating skin or hair, or both, comprising applying the composition described herein to the skin or hair or both. In addition, the present invention provides methods of controlling and/or preventing irritation, inflammation, immunosuppression or acne on skin or hair or a combination thereof, as well as ameliorating the signs of ageing and/or light-induced ageing of the skin or hair by applying an effective amount of the composition of the present invention, to skin or hair or both.

The present invention also provides kits or prepackaged materials containing the skin care compositions of the present invention. These kits or prepackaged materials can provide the retinoid formulations admixed directly into a skin care composition, provided separately, but in the same package as the skin care compositions, which then can be premixed and applied to the skin or hair; or applied sequentially. Likewise, the retinoid formulations can be provided in prepackaged kit coupled to one or more skin and/or hair treatment compositions (e.g., skin cleansers, skin astringents, hair cleansers, hair tonics, skin moisturizers, and others as described herein and known in the art).

Having generally described this invention, a further understanding can be obtained by reference to certain specific examples which are provided herein for purposes of illustration only, and are not intended to be limiting unless otherwise specified.

### EXAMPLES

### Example 1

0.15% Retinol Cream

| Phase | Component | Amount |
|---|---|---|
| A | Water | QA 100 g |
| | Preservative | 0.2 g |
| | Glycerin | 3 g |
| B | Preservative | 0.3 g |
| C | Simulgel 600 | 2.7 g |
| D | Isostearyl neopentanoate | 5 g |
| | stearylic alcohol | 1 g |
| E | triethanolamine | QS pH7 |
| | water | 10 g |
| F | retinol in soybean oil 10% | 1.5 g |

The formulation was prepared as follows:

1. Phase A was heated to 80 to 85° C. and degassed under vacuum;

2. While mixing in a Greerco homogenizer, Phase A was cooled to 45° C., the vacuum was removed, Phase B was added, the vacuum was reapplied and mixed until uniform;

3. At 45° C., the vacuum was removed, Phase C was added, the vacuum was reapplied, and mixed until uniform;

4. At 45° C., the vacuum was removed, Phase D was added, the vacuum was reapplied and mixed until uniform;

5. The batch was cooled to 30° C., the vacuum was removed, Phase E was added, the vacuum was reapplied, and mixed until uniform; and

6. The batch was cooled to 25° C., and under Argon gas and Actinic lights, Phase F was added and mixed until uniform.

### Example 2

0.3% Retinol Cream

| Phase | Component | Amount |
|---|---|---|
| A | water | QA 100 g |
| | preservative | 0.2 g |
| | glycerin | 3 g |
| B | preservative | 0.3 g |
| C | Simulgel 600 | 2.7 g |
| D | Isostearyl neopentanoate | 5 g |
| | stearylic alcohol | 1 g |
| B | triethanolamine | QS pH7 |
| | water | 10 g |
| F | retinol in soybean oil 10% | 3.0 g |

This formulation was prepared as described in Example 1.

### Example 3

0.6% Retinol Cream

| Phase | Component | Amount |
|---|---|---|
| A | water | QA 100 g |
| | preservative | 0.2 g |
| | glycerin | 3 g |
| B | preservative | 0.3 g |
| C | Simulgel 600 | 2.7 g |
| D | Neopentanoate isostearyle | 5 g |
| | stearylic alcohol | 1 g |
| E | triethanolamine | QS pH7 |
| | water | 10 g |
| F | retinol in soybean oil 10% | 6 g |

This formulation was prepared as described in Example 1.

### Example 4

The formulation in Example 3 was stored in closed containers for a period of time at 25° C. and 45° C. to assess the stability of the retinol in the formulation. At each time point a new container was sampled and the amount of retinol was measured as described in U.S. Pat. No. 6,080,393. The results are presented below:

| | % retinol in the | % in the formulation |
|---|---|---|
| Week | formulation at 25° C. | retinol at 45° C. |
| 0 | 0.59 | — |
| 1 | 0.57 | 0.57 |
| 2 | 0.63 | 0.61 |
| 4 | 0.57 | 0.57 |
| 8 | 0.57 | 0.57 |
| 12 | 0.58 | 0.55 |

The results present in the table above demonstrate that the formulation effectively stabilized the retinol at both temperatures even after 12 weeks, where 98.3% and 96.5% of the retinol remained in the formulations.

### Example 5

The formulation in Example 3 was stored in a closed tube for one month at 4° C. in a temperature control chamber to assess the stability of the retinol in the formulation. The tube was removed from the chamber once per week and squeezed during the study. At each time point a sample of the formulation was removed from the same tube and the amount of retinol present was measured as in Example 4. Measurements of retinol concentration in the tube were also performed at room temperature and 45° C. The results are presented below:

| | % retinol remaining in the formulation |
|---|---|
| Temperature ° C. | after 1 month |
| 4 | 0.57 |
| 4 | 0.56 |
| RT | 0.56 |
| RT | 0.55 |
| 45 | 0.55 |
| 45 | 0.56 |

The results present in the table above demonstrate that the formulation effectively stabilized the retinol at the various temperatures tested.

### Example 6

The formulation in Example 2 was stored in a closed container for a period of time at 25° C. and 45° C. to assess the stability of the retinol in the formulation. The results are presented below:

| | % retinol in the | % in the formulation |
|---|---|---|
| Week | formulation at 25° C. | retinol at 45° C. |
| 0 | 0.32 | — |
| 1 | 0.31 | 0.31 |
| 2 | 0.31 | 0.33 |
| 4 | 0.31 | 0.31 |
| 8 | 0.31 | 0.30 |
| 12 | 0.31 | 0.31 |

The results present in the table above demonstrate that the formulation effectively stabilized the retinol at both temperatures even after 12 weeks, where 96.9% and 100% of the retinol remained in the formulations.

### Example 7

A dispersion was prepared, to which one or more retinoids can be added, to increase the Carbomer/fatty alcohol levels to thicken the dispersion.

| Phase | Component | % | 1000 G |
|---|---|---|---|
| A | Water | 78.000 | 780.000 |
| | Glycerin | 3.000 | 30.000 |
| B | Pemulen TR-2 | 0.200 | 2.000 |
| | Carbopol 980 | 0.400 | 4.000 |
| C | Capric/Caprylic Triglycerides | 3.000 | 30.000 |
| | Isostearyl neopentanoate | 4.000 | 40.000 |
| | Meadowfoam seed oil | 3.000 | 30.000 |
| | Cetearyl alcohol | 1.000 | 10.000 |
| | Behenyl alcohol | 0.500 | 5.000 |
| | Preservative | 0.700 | 7.000 |
| | Water | 5.000 | 50.000 |
| D | Triethanolamine | 1.200 | 12.000 |
| | Total | 100.000 | 1000.000 |

1. Phase A was added in the main batch beaker, degassed under vacuum, and mixed until homogeneous;

2. Phases B, C, and D were added into separate beakers/containers;

3. Phase B was added slowly onto phase A and mixed with a propeller blade at 800 rpm until polymers were hydrated;

4. The batch was transferred to a Greerco Homogenizer;

5. The Main batch and phase C were heated to 80° C. with stirring;

6. Phase C was slowly added to the main batch while homogenizing and homogenization was continued for 5 minutes;

7. Phase D was added and homogenization was continued for 3 minutes;

8. The batch was cooled while mixing the batch with a Caframo mixer.

The properties of this formulation were:

Viscosity: 36,000 cps (Brookfield RVT; 10 rpm, spindleT-C, 1 min @ 25° C.)

pH @ 25° C.: 7.7

### Example 8

The purpose of this study was to determine by repetitive epidermal contact the potential of a test material to induce primary or cumulative irritation and/or allergic contact sensitization.

Induction Phase

A human repeat insult patch test was performed with the 0.6% formulation according to Example 3 on 203 human male and female test subjects from 18 to 70 years of age who did not have any visible skin disease which might be confused with a skin reaction from the test material. 0.2 g of sample was applied three times per week for a total of nine applications to the upper back between the scapule on a 1 in² absorbent pad of a clear adhesive dressing. The site was marked to insure continuity of patch applications. Following removal of the first induction patch after 24 hours, all subsequent patches were removed 24 hours after application.

Challenge Phase

Approximately two weeks after the final induction patch application, a challenge patch was applied to a virgin test site adjacent to the original induction patch site as described for the Induction phase.

The participants were evaluated for responses on a scale ranging from 0, no visible skin reaction, to 4, severe erythema, possible edema, vesiculation, bullae and/or ulceration.

While numerous, scattered barely perceptible responses were observed throughout the test interval, this type of reactivity is consistent with similar formulations evaluated under repetitive, semi-occlusive patch conditions. Under the results of this analysis, the test material did not indicate a clinically significant potential for dermal irritation or allergic contact sensitization.

### Example 9

The purpose of this study was to determine if repeated use of a facial skin treatment product according to Example 1, 0.15% retinol cream, elicited adverse reactions during normal use conditions.

Forty-eight adult females from ages 35 to 70 years of age completed the study, who had self-perceived sensitive skin and Fitzpatrick skin type I-IV. The subjects used the cream for four weeks without using other moisturizers, skin care products and make-up products. The subjects applied the cream every morning followed by the application of a sunscreen. The subjects were evaluated one, two and four weeks during the test period. Under the test conditions, the cream did not elicit clinically significant adverse reactions when subjected to four weeks of repetitive use.

### Example 10

The purpose of this study was to determine if repeated use of a facial skin treatment product according to Example 1, 0.15% retinol cream, elicited adverse reactions during normal use conditions.

Forty-six adult females from ages 35 to 69 years of age completed the study, which had Fitzpatrick skin type I-II. The subjects used the cream for four weeks without using other moisturizers, skin care products and make-up products. The subjects applied the cream every morning followed by the application of a sunscreen. The subjects were evaluated one, two and four weeks during the test period. Under the test conditions, the cream did not elicit clinically significant adverse reactions when subjected to four weeks of repetitive use.

### Example 11

The purpose of this study was to determine if repeated use of a facial skin treatment product according to Example 2, 0.3% retinol cream, elicited adverse reactions during normal use conditions.

Forty-nine adult females from ages 35 to 70 years of age completed the study, which had Fitzpatrick skin type III. The subjects used the cream for four weeks without using other moisturizers, skin care products and make-up products. The subjects applied the cream every morning followed by the application of a sunscreen. The subjects were evaluated one, two and four weeks during the test period. While there were some adverse experiences, these experiences are consistent with responses induced by similar retinol formulations. Therefore, under the test conditions, the cream did not elicit clinically significant adverse reactions when subjected to four weeks of repetitive use.

### Example 12

The purpose of this study was to determine if repeated use of a facial skin treatment product according to Example 3, 0.6% retinol cream, elicited adverse reactions during normal use conditions.

Fifty-two adult females from ages 35 to 69 years of age completed the study, which had Fitzpatrick skin type IV. The subjects used the cream for four weeks without using other moisturizers, skin care products and make-up products. The subjects applied the cream every morning followed by the application of a sunscreen. The subjects were evaluated one, two and four weeks during the test period. While there were some adverse experiences, these experiences are consistent with responses induced by similar retinol formulations. Therefore, under the test conditions, the cream did not elicit clinically significant adverse reactions when subjected to four weeks of repetitive use.

1. A composition comprising by weight relative to the total weight of the composition about 2.0% to about 7.0% Glycerin, about 0.5% to about 5.0% a homopolymer of acrylic acid, cross-linked with an allyl ether of an allyl ether of pentaerythritol, an allyl ether of sucrose, or an allyl ether of propylene; about 1.0% to about 7.0% Isostearyl neopentanoate; about 0.25% to about 2.0% Stearyl alcohol; and about 0.5% to about 6.0% retinoid.

2. The composition of claim 1, wherein the retinoid is water-soluble or oil-soluble.

3. The composition of claim 1, wherein the retinoid is selected from the group consisting of retinoic acid, a retinol aldehyde, and a retinol ester.

4. The composition of claim 1, wherein the retinoid is a retinol ester.

5. The composition of claim 4, wherein the retinol ester is selected from the group consisting of retinyl acetate, retinyl propionate, retinyl butylate, retinyl hexanoate, retinyl heptanoate, retinyl caprylate, and retinyl palmitate.

6. The composition of claim 1, wherein the retinoid is present in an amount of from 0.001 to 6% by weight relative to the total weight of the composition.

7. The composition of claim 1, wherein the retinoid is present in an amount of from 0.01 to 3% by weight relative to the total weight of the composition.

8. The composition of claim 1, wherein the retinoid is present in an amount of from 0.1 to 1% by weight relative to the total weight of the composition.

9. The composition of claim 1, wherein the retinoid is present in an amount of from 0.15 to 0.75% by weight relative to the total weight of the composition.

10. The composition of claim 1, wherein the retinoid is present in an amount of 0.15%, 0.3% or 0.6% by weight relative to the total weight of the composition.

11. The composition of claim 1, which retains at least about 90% of the retinoid after 12 weeks of storage at 45° C.

12. A method of making the composition of claim 1, comprising mixing the components.

13. The method of claim 12, wherein the components are mixed under an inert gas.

14. The method of claim 13, wherein the inert gas is argon gas.

15. In a method of providing a retinoid to skin, hair or both, the improvement comprising applying the composition of claim 1 to said skin, hair or both.

16. A composition comprising by weight relative to the total weight of the composition a retinoid, about 2.0% to about 7.0% Glycerin; about 0.05% to about 0.75% acrylates/C₁₀-C₃₀ alkyl acrylate copolymers; about 0.05% to about 0.75% a homopolymer of acrylic acid, cross-linked with an allyl ether of an allyl ether of pentaerythritol, an allyl ether of sucrose, or an allyl ether of propylene; about 1.0% to about 6.0% Capric/Caprylic Triglyceride; about 1.0% to about 8.0% Meadowfoam seed oil; about 0.25% to about 2.0% Cetearyl alcohol; about 0.25% to about 1.0% Behehyl alcohol; and about 0.25% to about 2.0% Triethanolamine.

17. The composition of claim 16, wherein the retinoid is water-soluble or oil-soluble.

18. The composition of claim 16, wherein the retinoid is selected from the group consisting of retinoic acid, a retinol aldehyde, and a retinol ester.

19. The composition of claim 16, wherein the retinoid is a retinol ester.

20. The composition of claim 16, wherein the retinol ester is selected from the group consisting of retinyl acetate, retinyl propionate, retinyl butylate, retinyl hexanoate, retinyl heptanoate, retinyl caprylate, and retinyl palmitate.

21. The composition of claim 16, wherein the retinoid is present in an amount of from 0.001 to 10% by weight relative to the total weight of the composition.

22. The composition of claim 16, wherein the retinoid is present in an amount of from 0.01 to 3% by weight relative to the total weight of the composition.

23. The composition of claim 16, wherein the retinoid is present in an amount of from 0.1 to 1% by weight relative to the total weight of the composition.

24. The composition of claim 16, wherein the retinoid is present in an amount of from 0.15 to 0.75% by weight relative to the total weight of the composition.

25. The composition of claim 16, wherein the retinoid is present in an amount of 0.15%, 0.3% or 0.6% by weight relative to the total weight of the composition.

26. The composition of claim 16, which retains at least about 90% of the retinoid after 12 weeks of storage at 45° C.

27. A method of making the composition of claim 16, comprising mixing the components.

28. The method of claim 27, wherein the components are mixed under an inert gas.

29. The method of claim 28, wherein the inert gas is argon gas.

30. In a method of providing a retinoid to skin, hair or both, the improvement comprising applying the composition of claim 16 to said skin, hair or both.

31. The composition of claim 16, which comprises by weight relative to the total weight of the composition 3.0% Glycerin; 0.2% of an acrylates/C₁₀-C₃₀ alkyl acrylate copolymers; 0.4% of a homopolymer of acrylic acid, cross-linked with an allyl ether of an allyl ether of pentaerythritol, an allyl ether of sucrose, or an allyl ether of propylene; 3.0% Capric/Caprylic Triglyceride; 3.0% Meadowfoam seed oil; 1.0% Cetearyl alcohol; 0.5% Behehyl alcohol; and 1.2% Triethanolamine.

32. The composition of claim 31, which comprises 0.15% retinoid by weight relative to the total weight of the composition.

33. The composition of claim 32, wherein the retinoid is retinol.

34. In a method of providing a retinoid to skin, hair or both, the improvement comprising applying the composition of claim 31 to said skin, hair or both.

35. In a method of providing a retinoid to skin, hair or both, the improvement comprising applying the composition of claim 32 to said skin, hair or both.

36. In a method of providing a retinoid to skin, hair or both, the improvement comprising applying the composition of claim 33 to said skin, hair or both.

37. The composition of claim 31, which comprises 0.3% retinoid by weight relative to the total weight of the composition.

38. The composition of claim 37, wherein the retinoid is retinol.

39. In a method of providing a retinoid to skin, hair or both, the improvement comprising applying the composition of claim 37 to said skin, hair or both.

40. In a method of providing a retinoid to skin, hair or both, the improvement comprising applying the composition of claim 38 to said skin, hair or both.

41. The composition of claim 31, which comprises 0.6% retinoid by weight relative to the total weight of the composition.

42. The composition of claim 41, wherein the retinoid is retinol.

43. In a method of providing a retinoid to skin, hair or both, the improvement comprising applying the composition of claim 41 to said skin, hair or both.

44. In a method of providing a retinoid to skin, hair or both, the improvement comprising applying the composition of claim 42 to said skin, hair or both.
